(19) United States
(12) Patent Application Publication
(10) Pub. No.: US 2018/0049652 A1
(43) Pub. Date: Feb. 22, 2018

(54) DIAGNOSTIC METHOD AND SYSTEM

(71) Applicant: United Arab Emirates University, Al Ain (AE)

(72) Inventors: Mahmoud Al Ahmad, Al Ain (AE); Mohamed Al Hemairy, Al Ain (AE); Saad Amin, Coventry (GB)

(21) Appl. No.: 15/383,481

(22) Filed: Dec. 19, 2016

Related U.S. Application Data
(60) Provisional application No. 62/377,223, filed on Aug. 19, 2016.

(51) Int. Cl. A61B 5/0205; A61B 5/00

(52) U.S. Cl. CPC A61B 5/02055; A61B 5/7232; A61B 5/024; A61B 5/7282; A61B 5/742

(57) ABSTRACT

Self-diagnosis of diseases is highly desired and very popular nowadays. The present application provides system, methodology, and the like for providing real-time detection of a medical condition.

### CROSS-REFERENCE TO RELATED APPLICATIONS

This application claims priority to U.S. Provisional Application No. 62/377,223, filed Aug. 19, 2016, the entire application hereby incorporated in its entirety.

### FIELD

The present disclosure relates to disease detection and related system and methodology.

### INTRODUCTION

Vital signs mainly body temperature, blood pressure, heart rate, and breathing rate are the most important signs that are commonly used to monitor human's body basic functions [1]. These indicators help in assessing the physical health of a person by providing diagnosis of possible diseases, and checking treatment progress towards recovery [2].

### SUMMARY

In one aspect, provided is a system, comprising:

one or more biometric sensors;

one or more processors coupled to the one or more biometric sensors; and

one or more storage devices storing instructions that are operable, when executed by the one or more processors, to cause the one or more processors to perform operations comprising:

obtaining data from the one or more biometric sensors, the data being captured by the one or more biometric sensors during a prior temporal interval;

establishing a weighting factor for each of the one or more biometric sensors;

calculating a control value based on the obtained data;

determining a value of an event indicator based on the obtained data, established weighting factor, and the calculated control value,

accessing a database that correlates corresponding event indicator values with candidate events;

based on the accessed database, establishing a match between the determined event indicator value and a corresponding one of the candidate events; and

performing one or more operations in accordance with the established correspondence.

In one embodiment, the candidate events comprises occurrences of at least one of a medical condition or a disease within a human population.

In another embodiment, the performing comprises: generating a signal indicative of the established correspondence, the generated signal being indicative of the match between the determined event indicator value and the corresponding candidate event; and transmitting the generated signal to an additional computing system, the signal instructing the additional computing system to modify a corresponding operational state and present at least one of a graphical, aural, or tactile alert.

In another embodiment, the performing comprises: generating a graphical representation of the match between the determined event indicator value and the corresponding candidate event; and presenting the generated graphical representation through a display unit.

In another aspect, the present disclosure provides a computer-implemented method, comprising:

receiving, by at least one processor, data from the one or more biometric sensors, the data being captured by the one or more biometric sensors during a prior temporal interval;

establishing, by the at least one processor, a weighting factor for each of the one or more biometric sensors;

calculating, by the at least one processor, a control value based on the obtained data;

determining, by the at least one processor, a value of an event indicator based on the obtained data, established weighting factor, and the calculated control value,

accessing, by the at least one processor, a database that correlates corresponding event indicator values with candidate events;

based on the accessed database, establishing, by the at least one processor, a match between the determined event indicator value and a corresponding one of the candidate events; and

performing, by the at least one processor, one or more operations in accordance with the established correspondence.

In another aspect, provided is a method for detecting a medical condition in a subject, comprising
(a) measuring body conditions using one or more sensors, wherein said sensor(s) is in contact with said subject's body;
(b) defining a weighting factor for each of the sensor;
(c) determining a Control value based on the measured sensor values;
(d) determining an indicator based on the weighting factor, Control value and the measured sensor values; and
(e) searching the value of the indicator in a look-up table to determine a medical condition (disease) corresponding the indicator.

### BRIEF DESCRIPTION OF THE DRAWINGS

FIG. 1: Workflow diagram illustrating an online system architecture. The workflow has four stages: a, b, c, and d.

FIG. 2: Exemplary platform for measuring biometrics.

FIG. 3: Exemplary medical condition detection system.

FIG. 4: Exemplary disease diagnosis system.

FIG. 5: Pseudocode for Disease Search Algorithm

FIG. 6: Exemplary eHealth test bench system.

FIG. 7: Exemplary wearable sensor simulator system.

FIG. 8: Exemplary evaluation system comprising a simulator, gateway, display, and server.

FIG. 9: Flowchart of data transfer from sensors simulator (Peripheral) to medical gateway (Central).

FIG. 10: Transfer time from medical gateway to server over several tests.

FIG. 11. Pseudocode for a Sequential Search Algorithm

FIG. 12: Comparison chart of disease detection time using exemplary eHealth system and lookup table.

FIG. 13: Real-time testing on server for performance in detecting diseases.

### DETAILED DESCRIPTION

Vital signs mainly body temperature, blood pressure, heart rate, and breathing rate are the most important signs that are commonly used to monitor human's body basic functions [1]. These indicators help in assessing the physical health of a person by providing diagnosis of possible diseases, and checking treatment progress towards recovery [2]. Table 1 below presents some common diseases [3] along with their corresponding medical conditions and sensors used to measure associated vital sign alteration. Table 1 also provides a brief description of each disease.

TABLE 1
Defined diseases and corresponding medical conditions

| Disease | Description | Vital signs ranges | Associated sensor/s |
|---|---|---|---|
| Bradycardia | abnormally slow heart rate | <60 beats/min | HR_SENSOR |
| Tachycardia | abnormally fast heart rate | >100 OR >120 beats/min | HR_SENSOR |
| Hypotension | abnormally low blood pressure | BP <100 mm Hg systolic | BP_SENSOR |
| Hypertension | abnormally high blood pressure | Mild to moderate (systolic | BP_SENSOR |
| | | blood pressure <180 mm | |
| | | Hg and diastolic blood | |
| | | pressure below 110 mm Hg) | |
| | | Severe hypertension, | BP_SENSOR |
| | | defined as a systolic | |
| | | pressure >180 mm Hg or | |
| | | diastolic pressure >110 mm Hg, | |
| Hypoxaemia | abnormally low concentration of oxygen | SPO2 <95% | SPO2_SENSOR |
| | in the blood. | | |
| Hyperthermia | abnormally high body temperature | core temperature >37.80° C. | TEMP_SENSOR |
| Hypothermia | Abnormally low body temperature | core temperature <36.0° C. | TEMP_SENSOR |
| Bradypnea | abnormally slow breathing rate | RR <20 breaths/min | RR_SENSOR |
| Tachypnea | abnormally fast breathing rate | RR >25 breaths/min | RR_SENSOR |
| Sinus | P waves are hidden within each preceding | ECG image “camel hump” | ECG_SENSOR |
| Tachycardia | T wave. | appearance | |
| Prediabetes | blood sugar level is higher than normal | Fasting glucose level: | GLOCOSE_SENSOR |
| | but not yet high enough to be classified as | (100-125) (mg/dL) | |
| | type 2 diabetes | | |
| Diabetes | describes a group of metabolic diseases in | Fasting glucose level: | GLOCOSE_SENSOR |
| | which the person has high blood glucose | more than 125 (mg/dL) | |
| | (blood sugar), either because insulin | | |
| | production is inadequate, or because the | | |
| | body's cells do not respond properly to | | |
| | insulin, or both | | |
| Pneumonia | a disease of the lungs characterized | RR >25 breaths/min | RR_SENSOR |
| | especially by inflammation and | HR >100 OR HR >120 beats/min | HR_SENSOR |
| | consolidation of lung tissue followed by | core temperature >37.80° C. | TEMP_SENSOR |
| | resolution and by fever, chills, cough, and | | |
| | difficulty in breathing and that is caused | | |
| | especially by infection | | |
| Urosepsis | is a systemic reaction of the body (SIRS) | core temperature >37.80° C. | TEMP_SENSOR |
| | to a bacterial infection of the urogenital | HR >100 or HR >120 beats/min | HR_SENSOR |
| | organs with the risk of life-threatening | BP <100 mm Hg systolic | BP_SENSOR |
| | symptoms including shock | | |
| Asthma Moderate | is a chronic inflammatory disorder of the | 90% < SPO2 <95% | SPO2_SENSOR |
| | airways | 100 < HR <120 beats/min | HR_SENSOR |
| | | RR >25 breaths/min | RR_SENSOR |
| Asthma Severe | is sever chronic inflammatory disorder of | SPO2 <90% | SPO2_SENSOR |
| | the airways | HR >120 beats/min | HR_SENSOR |
| | | RR >25 breaths/min | RR_SENSOR |
| Respiratory Arrest | is the cessation of normal breathing due | SPO2 <90% | SPO2_SENSOR |
| Imminent | to failure of the lungs to function effectively. | HR <60 beats/min | HR_SENSOR |
| | | RR >30 breaths/min | RR_SENSOR |

Detection and identification of diseases at early stage can facilitate the treatment significantly [4]. Unfortunately, due to the load of the daily work, most people do not find enough time to visit the doctor[5]. On the other hand, due to the frequent increment of diseases nowadays, it becomes impossible for the physicians to recall all symptoms and medical conditions for all kind of diseases [6]. Adequate assistive tools are necessary not only to help quickly identify the diseases but also to minimize the medical mistakes as well as to avoid prescribing invalid medications or treatments [7]. Online diagnosis system can be used to provide such diagnosis services [8]. The accurate detection and identification of a disease is highly dependable on the method used for diagnosis [9][10].

However, disease diagnosis is a very sophisticated process and demands high and advance level of expertise and it is an expensive process in terms of computational time and energy consumption [11]. Highly selective and efficient web-based clinical expert system is not yet developed in spite of the ongoing and existing trails and available systems [12]. Existing expert system incorporates inference rules [13][14][15]. Those rules play significant role in suggesting specific methods for disease diagnosis and treatment. Currently, there are several reports on e-health management systems that employ different diagnostic tools [16][17]. There is are ongoing scientific discussions and debate about which kind of diseases should be included in medical diagnosis expert system along with their symptoms [18][19]. Furthermore, which factors should be considered in diagnosis for such system, what approach should be followed [20], etc.

The present inventors provide system, methodology, and the like for diagnosing any kind of disease. In one embodiment, the present inventors developed a system comprising one or more computing devices configured to perform operations consistent with an algorithm that incorporates mathematical expressions which are used to determine a variable called an “Indicator” (also called “eHealth Indicator”) and its minimum and maximum interval values. The system then uses this “Indicator” value to search a look up table for the predefined corresponding disease. The instant system is experimented on various scenarios and a software simulator has been developed for evaluation and performance testing.

As detailed below, the present inventors developed a systematic procedure for self-diagnosis of diseases, using a support system developed and tested. The system may perform operations that detect potential occurrences of, and compute indicia of, several medical conditions. Each medical condition is associated with specific symptoms and signs that are mapped directly with several kinds of sensors and their readings. The instant disease diagnosis approach starts with reading the user real time vital signs using a wearable sensor system. Two variables have been introduced, the “control” to account for the sensor output range and whether it is normal or not and the “weighting factor” to determines the significant contribution of the corresponding sensor. As explained below, the present inventors developed an algorithm that incorporates new mathematical expressions which are used to determine the “Indicator” and its minimum and maximum interval values. The system then uses this “Indicator” value to search a predefined diseases look up table for the corresponding disease. This system helps in assessing the physical health of a person by providing diagnosis of possible diseases, and checking treatment progress toward recovery. Using the instant system and algorithm, medical condition detection is faster than traditional techniques. That is, the present inventors observed the performance of calculating the health Indicator is faster 10% to 48% than the sequential search method.

### A. System Architecture

In one embodiment, and as described below, the present inventors developed a system architecture that permits medical condition detection based on an Indicator value within minimum or maximum ranges of a defined medical condition. In some embodiments, for example, a system architecture has four stages: a pre-defined stage, pre-processing calculations stage, processing steps stage, and a medical condition's detection stage.

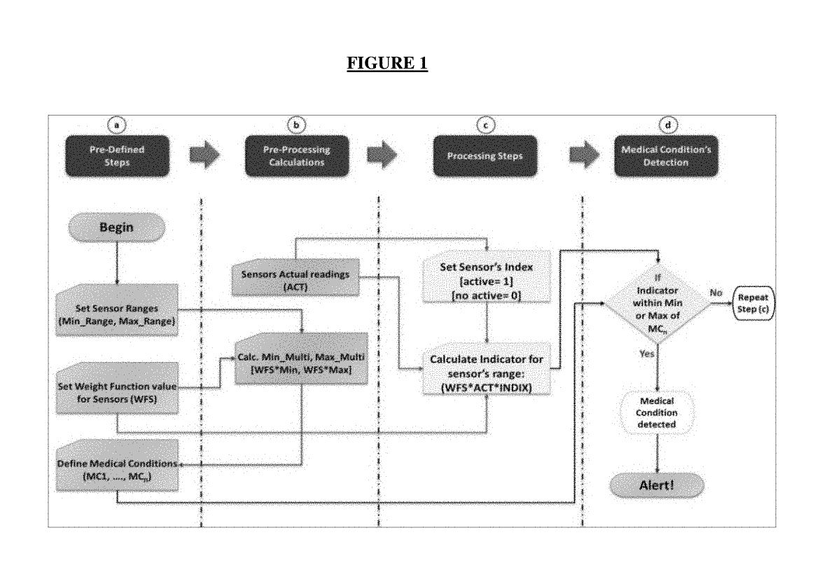

For instance, and as illustrated in FIG. 1, an illustrative system architecture may have four stages: (a) Pre-Defined stage, (b) Pre-Processing Calculations, (c) Processing operations, and (d) Medical Condition' Detection.

(a) Pre-Defined stage: where sensor ranges are setup with their corresponding min & max ranges, weighting Factor defined, as well as the medical conditions.

(b) Pre-Processing Calculations: in this stage, the sensors values are captured and stored, and the minimum multiplication for each sensor is calculated using the weight factor (WF) defined from the previous stage.

(c) Processing operations: set up the Control value for each sensor [0, 1] depending on its normal/abnormal value, the Indicator factor will also be calculated based on: WF, ACT & Control values.

(d) Medical Condition' Detection: this is the final stage where the medical condition is detected based on the Indicator value within the minimum or maximum ranges of the defined medical condition.

The system may perform operations that detect potential occurrences of, and compute indicia of several defined medical conditions. Usually a disease is constructed as a medical condition associated with specific symptoms and vital signs. Vital signs normally vary with, for example, age, weight, gender, and overall health [21] [22]. Measuring the vital signs for a person will provide an accurate figure about the body physical status and the health condition. Due to the technological advancement of the biological sensor, presently there are dedicated sensors for each vital sign to capture the corresponding vital sign [23]. Most of the regular diseases occurred for the human are related to the status of the vital signs and if their values are within or beyond the normal ranges. These vital signs are collected using dedicated sensors such as temperature, ECG, and breathing sensors.

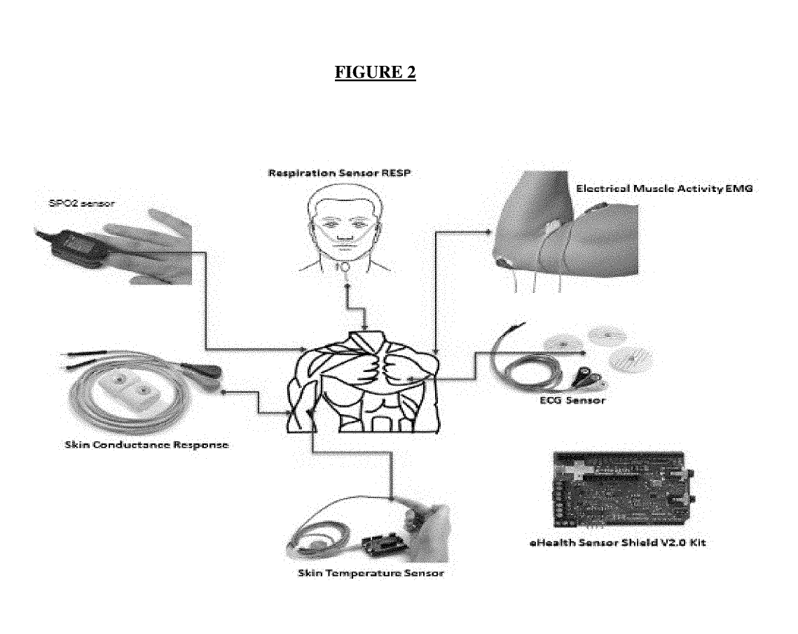

To accelerate development of a system architecture, the present inventors used a commercially available platform, namely e-Health Sensor Platform V2.0[24]. The platform consists of 9 different wearable sensors which measure 11 vital signs and a shield to connect the sensors. FIG. 2 illustrates the sensors and the shield. Of course, it is understood that a similar platform could be used, and the present disclosure in no way requires a specific platform or commercial product.

While in no way limiting, Table 2 below provides a brief description of 9 sensors and the biometrics they measure. The present system measures 11 different biological signals. Those 11 signals have normal ranges that if a value outside the normal range has been detected, then the physiological status of the person is abnormal and then probably has a medical condition. The ranges for these signals changes according to many factors such as, for example, age, location etc. For example, heart rate normal ranges for an infant if he is awake is between 100 and 190 beats per minute (bpm) but while he is sleeping the range becomes 90 to 160 bpm. On the other hand, a sleeping adult normal heart rate is between 50 and 90 bpm but if he is awake the range becomes 60 to 100 bpm [25].

TABLE 2
Wearable Health Sensors and the biometric they measure [26]

| The Sensor | Biometric it measures |
|---|---|
| Pulse and SPO2 sensor | Heart Rate (HR) |
| | Arterial oxygen saturation (SPO2) |
| Airflow sensor | Respiratory rates (RR) |
| Body temperature sensor | Body temperature (TEMP) |
| (ECG) sensor | Assess the electrical and muscular |
| | functions of the heart. |
| Glucometer | Approximate concentration of glucose |
| | in the blood |
| Sphygmomanometer | Systolic blood pressure (SBP) |
| | Diastolic blood pressure (DBP) |
| Galvanic skin response | Measuring electrical conductance of the |
| sensor (GSR) | skin, which varies with its moisture level |
| Accelerometer | Patient positions |
| Muscle/electromyography | Electrical activity of muscles |
| sensor (EMG) | |

The instant system may store, in one or more tangible, non-transitory memories, structured data records (e.g., within a lookup database) that facilitate a detection of a particular medical condition based on biometric and other data captured by wearable devices in communication with the system across one or more communications networks.

### B. Medical Condition Detection

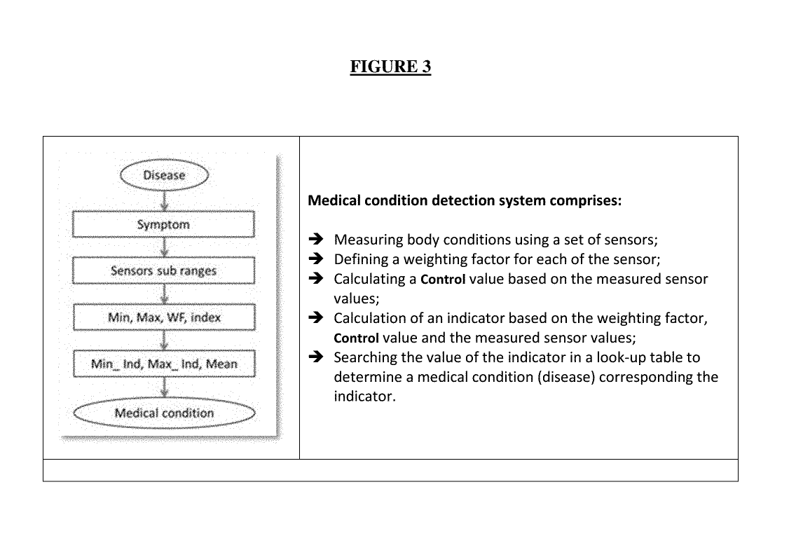

FIG. 3 describes a medical condition detection system, which includes detecting a medical condition, or a disease from a list of defined medical conditions (diseases) based on the calculation of a variable called an Indicator. First, a disease must be identified. Second, the symptoms of the disease should be specified. Third, the involved sensors sub ranges are defined. Forth, the maximum and the minimum value for the involved sensor are established and the corresponding control value for the involved sensors will be set to ‘1’. A weighting factor (WF) value is introduced. The weighting factor is a unique value assigned to each sensor. This value determines the significant contribution of the corresponding sensor. The WF value varies from “0” to “1”. The weighting factor value corresponds to the frequency of use of a specific kind of sensor in several medical conditions. In other words, for example, if there are 100 defined medical conditions based on 10 kind of sensors readings and the temperature is included in all of them, then its corresponding WF is 1, and if it is included in 85 conditions, its WF is 0.85 and so on and so forth. This factor will be used later in the computation of the “Indicator” value used to identify the corresponding medical condition. Since the WF depends on the total number of defined disease in our database, every time we add a new disease we update the WF for the involved sensors. Fifth, the maximum, minimum “Indicator” value for the disease is computed and attached to the corresponding medical condition in the disease lookup table.

For instance, Table 3 below shows the weighting factors for some of the sensors used according to the defined medical conditions, consistent with disclosed embodiments. In some aspects, certain of the disclosed systems may store one or more weighting factors in a corresponding database. The weighting factors numbers assigned to different type of sensors are listed in Table 3.

TABLE 3
Sample of the used sensors with their
corresponding weighting factor

| WFS | Sensor type | sensor Abbreviation |
|---|---|---|
| 0.7 | Heart Rate Sensor | HR_SENSOR |
| 0.9 | Blood Pressure Sensor | BP_SENSOR |
| 0.2 | Spo2 Sensor | SPO2_SENSOR |
| 0.6 | Temperature SENSOR | TEMP_SENSOR |
| 0.5 | Respiration Rate Sensor | RR_SENSOR |
| 0.2 | Glucose Level SENSOR | GLOCOSE_SENSOR |

It is noted that each sensor has a sensing range. This sensing range could be divided into small ranges. As an example, Table 4 below presents the sub ranges for human temperature sensor's reading as an example. In this example, the sensor has four (4) intervals each with its corresponding range values. When body temperature falls below 35.0° C., the subject has hypothermia. Hypothermia is a medical emergency that occurs when human's body loses heat faster than it can produce heat, causing a dangerously low body temperature. The normal range of internal human body temperature varies between (36.5-37.5)° C.

TABLE 4
Defined human temperature classification ranges [27][28][29][30][31]

| Ranges | Symptom | Interval |
|---|---|---|
| STR1 | Hypothermia | <35.0° C. (95.0° F.) |
| STNR | Normal | 36.5-37.5° C. (97.7-99.5° F.) |
| STR2 | Fever | >37.5 or 38.3° C. (99.5 or 100.9° F.) |
| STR4 | Hyperpyrexia | >40.0 or 41.5° C. (104.0 or 106.7° F.) |

### “Indicator” Computational Algorithm

As stated previously, the proposed system starts whenever subject sensors measurements are available. For each sensor, three parameters were defined, namely their WF, minimum and maximum values. The proposed system then uses these values to compute the corresponding minimum and maximum range values of the “Indicator” parameter. Table 4 is updated by adding to it a new column that represents the actual measured value. If the actual measured value lies in the normal range, the corresponding control value is set to “0”, otherwise to “1”. Based on this if all the sensors readings are within their normal ranges, then the ‘Indicator’ value will be “0”, as it will be shown later, thus no medical condition is detected (diseases free case). Table 5 shows the indicator computation matrix.

TABLE 5
Indicator computation matrix

| Sensor rule | WF | Min | Max | Actual | Control |
|---|---|---|---|---|---|
| S1R | WF1 | Min1 | Max1 | A1 | C1 = “0” or “1” |
| S2R | WF2 | Min2 | Max2 | A2 | C2 = “0” or “1” |
| S3R | WF3 | Min3 | Max3 | A3 | C3 = “0” or “1” |
| S4R | WF4 | Min4 | Max4 | A4 | C4 = “0” or “1” |
| S5R | WF5 | Min5 | Max5 | A5 | C5 = “0” or “1” |
| S6R | WF6 | Min6 | Max6 | A6 | C6 = “0” or “1” |
| S7R | WF7 | Min7 | Max7 | A7 | C7 = “0” or “1” |

The developed algorithm incorporates mathematical expressions, which are used to determine the “Indicator” and its minimum and maximum interval values. The system is then uses this Indicator value to search a look up table for the corresponding disease. The Indicator for a specific disease is computed using the below formula:

Indicator=Σᵢ₌₁¹²(WFᵢ)(Aᵢ)(Cᵢ) (1)

and the corresponding minimum and maximum for the indicator values for a specific disease are computed using the following equations:

Min_Ind=Σᵢ₌₁¹²(WFᵢ)(Minᵢ)(Cᵢ) (2)

Max_Ind=Σᵢ₌₁¹²(WFᵢ)(Maxᵢ)(Cᵢ) (3)

where WFᵢ, Aᵢ, Cᵢ, Minᵢ, Maxᵢ, and i are the weighting factor, actual reading of the sensor, control, minimum, maximum range values and the number of the sensor, respectively.

The Min_Ind and the Max_Ind values are computed and saved in disease lookup table. Each disease has an interval to identify it and this interval is defined by the Min_Ind and the Max_Ind values. Every time a new disease is added to a database its ‘Indicator’ interval is defined using formula 2 and 3. The disease lookup table is implemented as a binary search tree (BST). BST facilitate and accelerate range search process.

### Exemplary Computer-Implemented Processes for Automatic Disease Detection

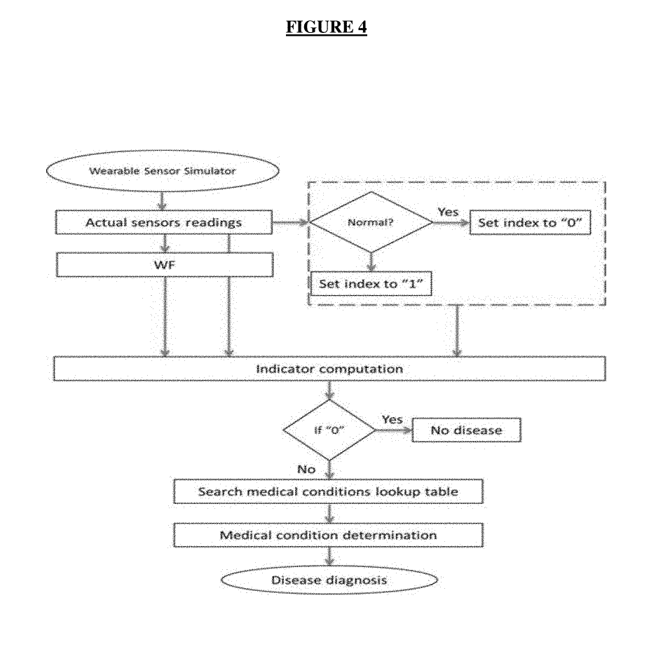

The detailed disease diagnosis overview is shown in FIG. 4. First, the user vital signs readings are provided to the system. The sensors whose readings are in the normal range their index (control) value will be set to zero and the other sensors control value will be set to 1. Then, the ‘Indicator’ value is computed from the actual sensor reading value, the sensor control value and the sensor weight factor value. If the computed ‘Indicator’ value equal zero then the user's vital signs are in the normal range but if the ‘Indicator’ value is greater than zero this mean the user is suffering from a specific disease. The ‘Indicator’ value is then used to search the disease lookup table for the corresponding disease and present it as the suggested diagnosis.

In no way limiting and as an example, Table 6 below shows the structure of the disease lookup table for four medical conditions. As revealed through equations (2) and (3), the calculation of the corresponding disease's minimum and maximum “Indicator” values is independent of the actual real time sensor reading. Indeed all the parameters used for determining Min_Ind and Max_Ind are predefined values.

TABLE 6
Disease lookup table for diagnosis and identification.

| Disease | Min_Ind | Max_Ind |
|---|---|---|
| MC1 | Min_Ind1 | Max_Ind1 |
| MC2 | Min_Ind2 | Max_Ind2 |
| MC3 | Min_Ind3 | Max_Ind3 |
| MC4 | Min_Ind4 | Max_Ind4 |

The instant system does not require any medical information to be provided and entered by the user manually. Rather, all what is needed is to connect the sensors to the subject's body. This may require a one-time training for the user to teach him/her where and how to place the sensors. The instant system, and certain exemplary computer-implemented processes described above, may be implement in addition to, or as an alternate to, web-based medical diagnostic tools where the user needs to type his symptoms manually given then the fact that he knows the medical term for the symptoms and their correct spelling, and diagnostic tools incapable of identifying symptomless diseases, such as Hypertension.

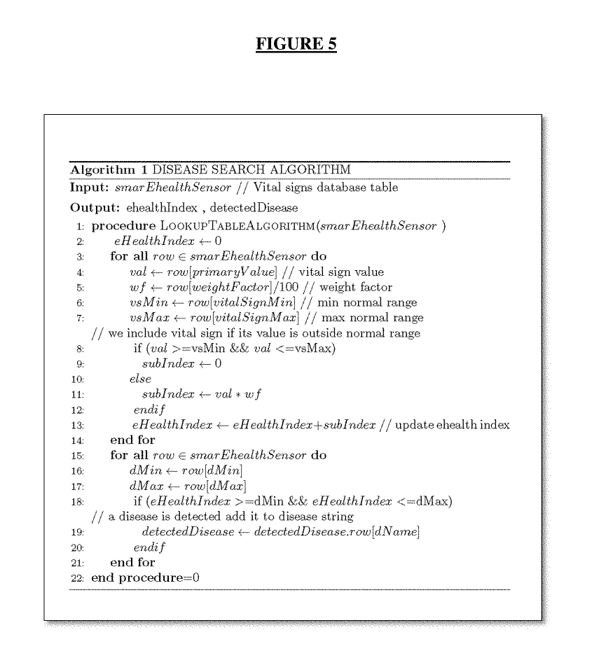

The process of detecting diseases using the new algorithm is depicted as a pseudocode, as exemplified in FIG. 5. [33]

### EXAMPLES

### System Testing and Evaluation

In order to demonstrate the applicability of the instant system and algorithm in real life situations, the inventors developed the main functions and components and performed various experimental tests. After that, the inventors conducted several measurements to evaluate system's performance.

It is understood that the below Examples are illustrative and non-limiting. It will, however, be evident that various modifications and changes may be made thereto, and additional embodiments may be implemented, without departing from the broader scope of the disclosed embodiments.

### Example 1

### System Testing Setup

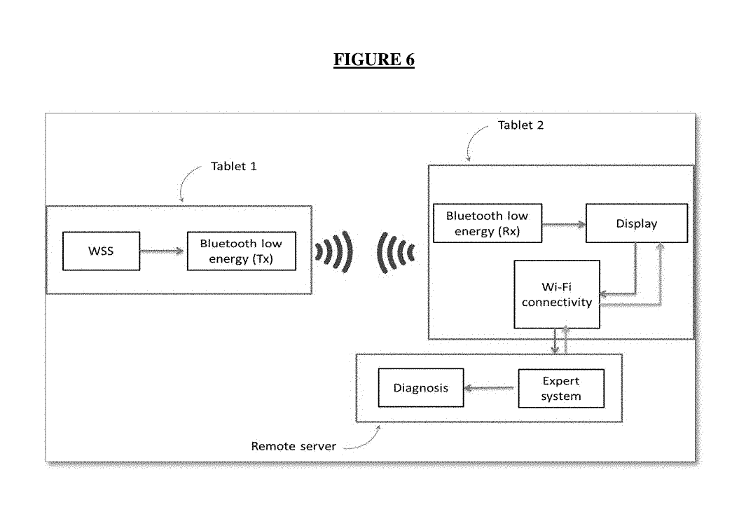

To validate the instant eHealth architecture and disease detection algorithm, the inventors developed a test bench, as shown in FIG. 6. The test bench has three elements: wearable Bluetooth sensors simulator, the medical gateway, and the eHealth remote server.

The simulator enable the simulation of various medical sensors output. This simulator will be installed on a tablet. In the actual system, the simulator will be replaced by a set of wearable medical sensors mounted on the patient (as depicted in FIG. 2). Digital values of vital signs are sent from the simulator to the medical gateway using Bluetooth low energy wireless network technology. The medical gateway (an Android application running on a smart tablet) collects vital signs and display them in real time; at the same time these values are transferred to eHealth server for further analysis and disease detection. The eHealth server analyzes vital signs values using the instant algorithm for disease detection (explained in the previous section). Once a disease or some symptoms have been detected, the server sends a notification to the patient, (this notification will be displayed in real time on the tablet) and an email alert will be sent to the doctor.

A software simulator with a set of virtual wearable sensors was designed to setup a specific medical condition. The simulator set of virtual sensors' output is adjustable and can be manipulated to correspond to a specific disease.

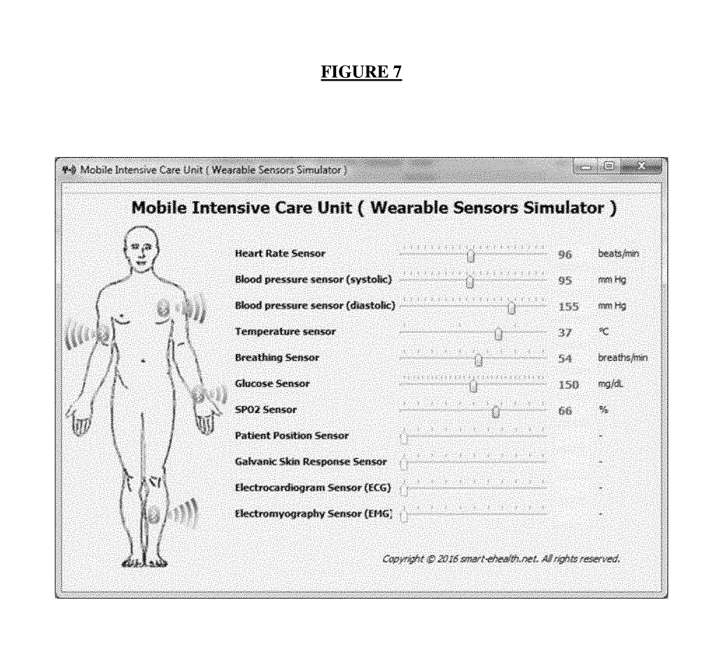

FIG. 7 provides an exemplary designed simulator. This hybrid simulator sensors configuration framework is developed to simulate continuous dynamics of the human's physiology. The medical conditions can be simulated by adjusting the slider to a certain value. A decision was made during the conducted experiments to only use the first seven sensors. The remaining sensors can be activated whenever there is a need. The listed medical conditions in Table 1 can be simulated by configuring the first seven sensors only and the simulator can be updated to include further type of sensors.

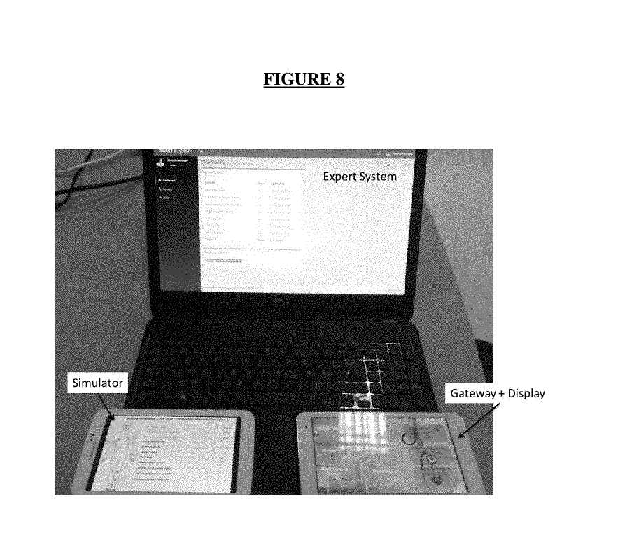

Different communication protocols are used to transmit the collected data to the storage and processing servers, i.e. Bluetooth Smart Ready and WiFi. The Bluetooth protocol was used because of its short-range connectivity, low power consumption, high connectivity bandwidth and its lightweight receiver/transmitter load. While the WiFi protocol was used to connect the gateway with the cloud servers via the internet due to its liability, and wide-range (approx. 50 m) connectivity, the cloud environment was chosen due to its availability, huge processing capabilities as well as its large storage resources. The test bed for the experimental setup is depicted in FIG. 8. The purpose of the experiment is to evaluate the performance of the instant algorithm in detecting the medical conditions. Those tests should demonstrate the efficiency of the instant algorithm in comparison with conventional and linear algorithms. The experiments should also evaluate system performance in terms of the data transfer rate and computational time.

### Example 2

### Bluetooth Data Transfer Time

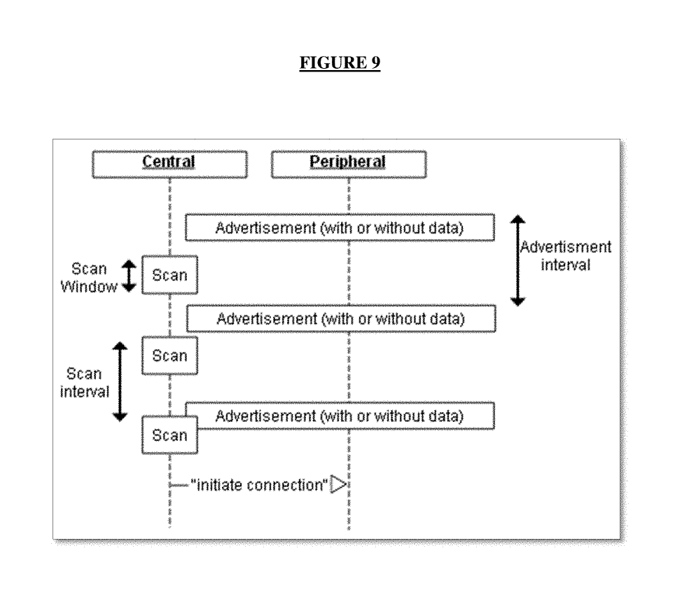

FIG. 9 displays the data transfer from the sensors simulator (Peripheral Device) to the medical gateway (called Central). The peripheral has an advertisement interval of 300 milliseconds (ms), however the advertisement time was fixed by the software to 100 ms. The Central has a scan window of 50 ms and a scan interval of 100 ms. Of course, it is understood that this is a non-limiting example.

### Example 3

### Data Transfer from Gateway to Server

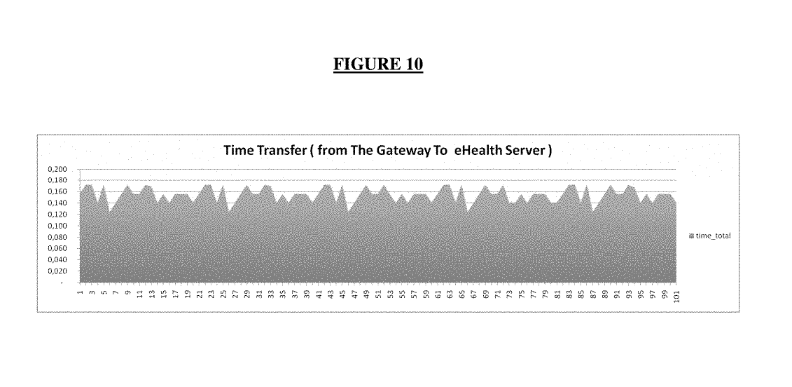

The second test will evaluate the data transfer time needed for sending data from medical gateway to server. The result are depicted in FIG. 10 and it shows an average of 155 milliseconds. The x axies represents number of tests run and the y axis represent the time in milliseconds.

### Example 4

### Testing Disease Detection Algorithm

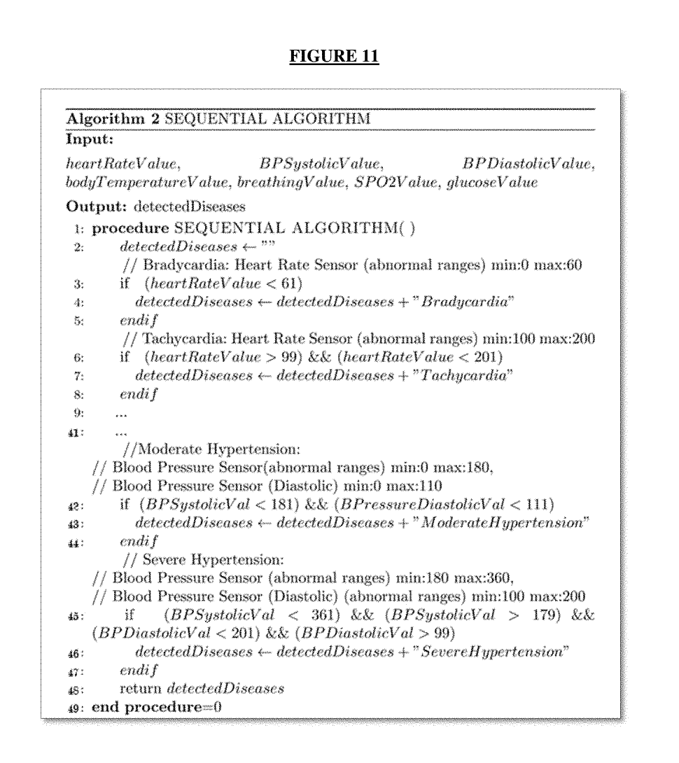

The last test was mainly designed to evaluate the performance and efficiency of the instant algorithm to detect disease. To measure the time required for disease detection a custom script was created, similar to the one executed on eHealth server to measure the differences between the proposed algorithm and any conventional algorithm using searching in a normal lookup table sequential as shown in the pseudocode below in FIG. 11.

The script includes measurement functions that measures the times required to execute the following tasks:
eHealth Indicator calculation time.
Disease search time in the lookup table.
Disease total detection time using eHealth Indicator and lookup table.
Disease detection time using the conventional sequential algorithm [32] (vital signs are compared with the normal and abnormal ranges of each sensor)

TABLE 7
Summary of the computation time lapsed to obtain from the different tests (time in seconds)

| | | | Time | Disease | Disease | Delta | |
|---|---|---|---|---|---|---|---|
| | | eHealth | to search | Detection | detection | time % | |
| | eHealth | Indicator | disease | time using | time using | Δ = | |
| TEST | Indicator | Calc. time | in lookup | Indicator | sequential | (D * 100/ | |
| No | value | (A) | (B) | C = A + B | test (D) | C) − 100 | Detected diseases |
| 1 | 376.900 | 0.000898 | 0.00015800 | 0.001056 | 0.001527 | 44.63% | Severe Hypertension |
| 2 | 48.500 | 0.000786 | 0.00011500 | 0.000901 | 0.001184 | 31.42% | Hypotension/Diabetes/ |
| | | | | | | | Moderate Hypertension |
| 3 | 176.400 | 0.000786 | 0.00011500 | 0.000901 | 0.001184 | 31.42% | Asthma Severe/Moderate |
| | | | | | | | Hypertension |
| 4 | 189.200 | 0.000816 | 0.00021900 | 0.001035 | 0.001165 | 12.53% | Asthma Severe/Moderate |
| | | | | | | | Hypertension |
| 5 | 0.000 | 0.001005 | 0.00015300 | 0.001158 | 0.001534 | 32.43% | //no disease detection |
| 6 | 111.400 | 0.000473 | 0.00008300 | 0.000556 | 0.000795 | 43.04% | Tachycardia/Asthma Severe/ |
| | | | | | | | Moderate Hypertension |
| 7 | 132.300 | 0.000804 | 0.00014800 | 0.000952 | 0.001305 | 37.10% | Tachycardia/Asthma Severe/ |
| | | | | | | | Moderate Hypertension |
| 8 | 21.700 | 0.000816 | 0.00021900 | 0.001035 | 0.001165 | 12.53% | Bradycardia/Hypotension/ |
| | | | | | | | Respiratory Arrest Imminent/ |
| | | | | | | | Moderate Hypertension |
| 9 | 35.000 | 0.000688 | 0.00014500 | 0.000833 | 0.001235 | 48.26% | Bradycardia/Hypotension/ |
| | | | | | | | Prediabetes/Respiratory Arrest |
| | | | | | | | Imminent/Moderate Hypertension |
| 10 | 111.300 | 0.000898 | 0.00018400 | 0.001082 | 0.001252 | 15.69% | Tachycardia/Asthma Severe/ |
| | | | | | | | Moderate Hypertension |
| 11 | 133.000 | 0.002494 | 0.00012700 | 0.002621 | 0.002945 | 12.37% | Tachycardia/Asthma Severe/ |
| | | | | | | | Moderate Hypertension |
| 12 | 221.000 | 0.000461 | 0.00007900 | 0.000540 | 0.000632 | 17.09% | Moderate Hypertension |
| 13 | 53.800 | 0.000868 | 0.00024200 | 0.001110 | 0.001351 | 21.67% | Hypotension/Diabetes/ |
| | | | | | | | Moderate Hypertension |
| 14 | 64.200 | 0.000919 | 0.00015000 | 0.001069 | 0.001561 | 46.00% | Hypotension/Diabetes/ |
| | | | | | | | Moderate Hypertension |
| 15 | 94.800 | 0.000847 | 0.00015600 | 0.001003 | 0.001403 | 39.93% | Tachycardia/Asthma Moderate/ |
| | | | | | | | Moderate Hypertension |
| 16 | 71.400 | 0.000873 | 0.00019300 | 0.001066 | 0.001245 | 16.80% | Tachycardia/Hypotension/ |
| | | | | | | | Diabetes/Moderate Hypertension |
| 17 | 30.500 | 0.000889 | 0.00019500 | 0.001084 | 0.001554 | 43.32% | Bradycardia/Hypotension/ |
| | | | | | | | Prediabetes/Respiratory Arrest |
| | | | | | | | Imminent/Moderate Hypertension |
| 18 | 376.900 | 0.000994 | 0.00019600 | 0.001190 | 0.001434 | 20.49% | Severe Hypertension |
| 19 | 48.500 | 0.000899 | 0.00016300 | 0.001062 | 0.001190 | 12.02% | Hypotension/Diabetes |
| | | | | | | | Moderate Hypertension |
| 20 | 21.700 | 0.000559 | 0.00010300 | 0.000662 | 0.000733 | 10.66% | Bradycardia/Hypotension/ |
| | | | | | | | Respiratory Arrest Imminent/ |
| | | | | | | | Moderate Hypertension |
| 21 | 166.500 | 0.000873 | 0.00019300 | 0.001066 | 0.001245 | 16.80% | Asthma Severe/Moderate |
| | | | | | | | Hypertension |
| 22 | 8.800 | 0.005223 | 0.00020700 | 0.005430 | 0.006163 | 13.49% | Bradycardia/Hypotension/ |
| | | | | | | | Hypoxaemia/Tachypnea/ |
| | | | | | | | Moderate Hypertension |
| 23 | 195.300 | 0.000780 | 0.00013800 | 0.000918 | 0.001296 | 41.15% | Asthma Severe/Moderate |
| | | | | | | | Hypertension |
| 24 | 241.300 | 0.001247 | 0.00021000 | 0.001457 | 0.001674 | 14.89% | Moderate Hypertension |
| 25 | 221.000 | 0.000461 | 0.00007900 | 0.000540 | 0.000775 | 43.43% | Moderate Hypertension |
| 26 | 221.000 | 0.000878 | 0.00015100 | 0.001029 | 0.001284 | 24.74% | Moderate Hypertension |
| 27 | 264.600 | 0.000878 | 0.00015100 | 0.001029 | 0.001284 | 24.74% | Severe Hypertension |

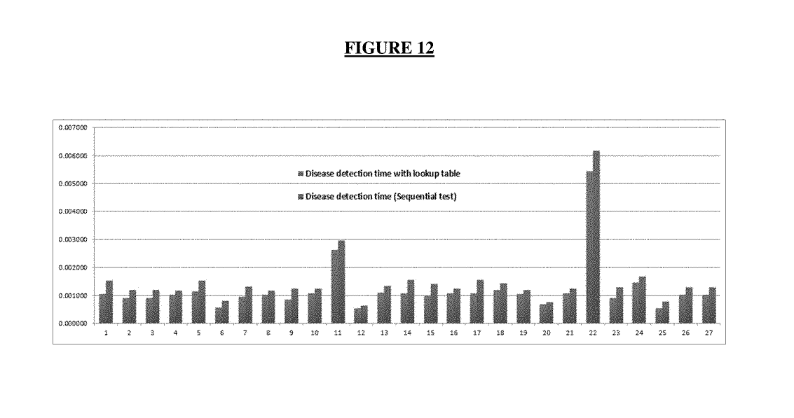

The comparison chart shown below (FIG. 12): shows that the disease detection time using the eHealth Indicator and the lookup table. It is clear that the instant algorithm is much faster than the conventional algorithm using the sequential test. The instant algorithm uses an access to the database in order to get real-time vital signs and to check the medical conditions, the calculation time change depending on the server load. Therefore, the tests were conducted on a dedicated local host instead of cloud-based server to avoid the server load factor. During all the tests conducted, it was observed that the performance of the method and system used in the instant algorithm for calculating the health Indicator is faster 10.66% to 48.26% than the sequential search method [32]

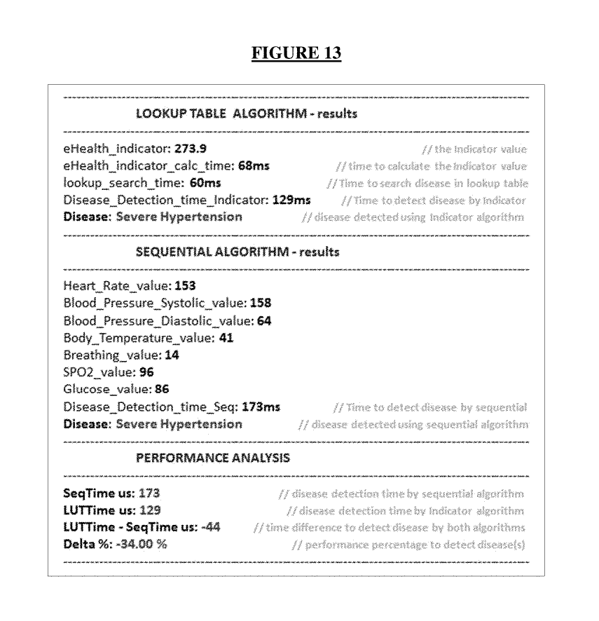

Compared to the conventional linear search (sequential search) method for finding the target rule in a list and trigger its action, the sequential search method checks each and every rule in the list until it finds the matching rule or all the rules are searched without finding a match. An online tool has been developed to test the instant algorithm's performance on real-time in detecting the diseases and improve the performance as fast as possible. FIG. 13 provides a screenshot of the online test.

For example to detect a “Severe Hypertension” by both algorithms based on the given vital signs by the sensors, the sequential “Serial” search algorithm elapsed 173 milliseconds to detect the disease, while the Indicator algorithm will need only 129 milliseconds to detect the same. This raises the performance to up to 34% for this particular medical condition. Further examples of diagnostic indicators are shown in the following Table 8.

TABLE 8
Further examples of diagnostic indicators

| Disease | Description | Vital signs ranges | Associated sensor/s |
|---|---|---|---|
| Bradycardia | abnormally slow heart rate | <60 beats/min | HR_SENSOR |
| Tachycardia | abnormally fast heart rate | >100 OR >120 beats/min | HR_SENSOR |
| Hypotension | abnormally low blood pressure | BP < 100 mm Hg systolic | BP_SENSOR |
| Hypertension | abnormally high blood pressure | Mild to moderate (systolic | BP_SENSOR |
| | | blood pressure <180 mm | |
| | | Hg and diastolic blood | |
| | | pressure below 110 mm Hg) | |
| | | Severe hypertension, | BP_SENSOR |
| | | defined as a systolic | |
| | | pressure >180 mm Hg or | |
| | | diastolic pressure >110 mm Hg, | |
| Hypoxaemia | abnormally low concentration of oxygen | SPO2 <95% | SPO2_SENSOR |
| | in the blood. | | |
| Hyperthermia | abnormally high body temperature | core temperature >37.80° C. | TEMP_SENSOR |
| Hypothermia | Abnormally low body temperature | core temperature <36.0° C. | TEMP_SENSOR |
| Bradypnea | abnormally slow breathing rate | RR <20 breaths/min | RR_SENSOR |
| Tachypnea | abnormally fast breathing rate | RR >25 breaths/min | RR_SENSOR |
| Sinus | P waves are hidden within each preceding | ECG image “camel hump” | ECG_SENSOR |
| Tachycardia | T wave. | appearance | |
| Prediabetes | blood sugar level is higher than normal | Fasting glucose level: | GLOCOSE_SENSOR |
| | but not yet high enough to be classified as | (100-125) (mg/dL) | |
| | type 2 diabetes | | |
| Diabetes | describes a group of metabolic diseases in | Fasting glucose level: | GLOCOSE_SENSOR |
| | which the person has high blood glucose | more than 125 (mg/dL) | |
| | (blood sugar), either because insulin | | |
| | production is inadequate, or because the | | |
| | body's cells do not respond properly to | | |
| | insulin, or both | | |
| Pneumonia | a disease of the lungs characterized | RR >25 breaths/min | RR_SENSOR |
| | especially by inflammation and | HR >100 OR HR >120 beats/min | HR_SENSOR |
| | consolidation of lung tissue followed by | core temperature >37.80° C. | TEMP_SENSOR |
| | resolution and by fever, chills, cough, and | | |
| | difficulty in breathing and that is caused | | |
| | especially by infection | | |
| Urosepsis | is a systemic reaction of the body (SIRS) | core temperature >37.80° C. | TEMP_SENSOR |
| | to a bacterial infection of the urogenital | HR >100 or HR >120 beats/min | HR_SENSOR |
| | organs with the risk of life-threatening | BP <100 mm Hg systolic | BP_SENSOR |
| | symptoms including shock | | |
| Asthma | is a chronic inflammatory disorder of the | 90% < SPO2 <95% | SPO2_SENSOR |
| Moderate | airways | 100 < HR <120 beats/min | HR_SENSOR |
| | | RR >25 breaths/min | RR_SENSOR |
| Asthma Severe | is sever chronic inflammatory disorder of | SPO2 <90% | SPO2_SENSOR |
| | the airways | HR >120 beats/min | HR_SENSOR |
| | | RR >25 breaths/min | RR_SENSOR |
| Respiratory | is the cessation of normal breathing due | SPO2 <90% | SPO2_SENSOR |
| Arrest Imminent | to failure of the lungs to function effectively. | HR <60 beats/min | HR_SENSOR |
| | | RR >30 breaths/min | RR_SENSOR |

### Exemplary Hardware and Software Implementations

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification, can be implemented as one or more computer programs, i.e., one or more modules of computer program instructions encoded on a tangible non transitory program carrier for execution by, or to control the operation of, data processing apparatus. Additionally or alternatively, the program instructions can be encoded on an artificially generated propagated signal, such as a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, such as an FPGA (field programmable gate array) or an ASIC (application specific integrated circuit). The apparatus can optionally include, in addition to hardware, code that creates an execution environment for computer programs, such as code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand alone program or as a module, component, subroutine, or other unit suitable for use in a computing environment. A computer program may, but need not, correspond to a file in a file system. A program can be stored in a portion of a file that holds other programs or data, such as one or more scripts stored in a markup language document, in a single file dedicated to the program in question, or in multiple coordinated files, such as files that store one or more modules, sub programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, such as an FPGA (field programmable gate array) or an ASIC (application specific integrated circuit).

Computers suitable for the execution of a computer program include, by way of example, general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, such as magnetic, magneto optical disks, or optical disks. However, a computer need not have such devices. Moreover, a computer can be embedded in another device, such as a mobile telephone, a personal digital assistant (PDA), a mobile audio or video player, a game console, a Global Positioning System (GPS) receiver, or a portable storage device, such as a universal serial bus (USB) flash drive, to name just a few.

Computer readable media suitable for storing computer program instructions and data include all forms of non volatile memory, media and memory devices, including by way of example semiconductor memory devices, such as EPROM, EEPROM, and flash memory devices; magnetic disks, such as internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, such as a CRT (cathode ray tube) or LCD (liquid crystal display) monitor, for displaying information to the user and a keyboard and a pointing device, such as a mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Implementations of the subject matter described in this specification can be implemented in a computing system that includes a back end component, such as a data server, or that includes a middleware component, such as an application server, or that includes a front end component, such as a client computer having a graphical user interface or a Web browser through which a user can interact with an implementation of the subject matter described in this specification, or any combination of one or more such back end, middleware, or front end components. The components of the system can be interconnected by any form or medium of digital data communication, such as a communication network. Examples of communication networks include a local area network (LAN) and a wide area network (WAN), such as the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, such as an HTML page, to a user device, such as for purposes of displaying data to and receiving user input from a user interacting with the user device, which acts as a client. Data generated at the user device, such as a result of the user interaction, can be received from the user device at the server.

While this specification contains many specifics, these should not be construed as limitations, but rather as descriptions of features specific to particular embodiments. Certain features that are described in this specification in the context of separate embodiments may also be implemented in combination in a single embodiment. Conversely, various features that are described in the context of a single embodiment may also be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Various embodiments have been described herein with reference to the accompanying drawings. It will, however, be evident that various modifications and changes may be made thereto, and additional embodiments may be implemented, without departing from the broader scope of the disclosed embodiments.

Further, other embodiments will be apparent to those skilled in the art from consideration of the specification and practice of one or more embodiments of the present disclosure. It is intended, therefore, that this disclosure and the examples herein be considered as exemplary only.

### REFERENCES

[1] L. Gatzoulis and I. Iakovidis, “Wearable and Portable eHealth Systems,” IEEE Eng. Med. Biol. Mag., vol. 26, no. 5, pp. 51-56, September 2007.
[2] S. Borson, J. Scanlan, M. Brush, P. Vitaliano, and A. Dokmak, “THE MINI-COG: A COGNITIVE VITAL SIGNSMEASURE FOR DEMENTIA SCREENING IN MULTI-LINGUAL ELDERLY,” Int. J. Geriatr. PSYCHIATRY Int. J. Geriatr Psychiatry, 2000.
[3] M. W. Papadakis, M. A., McPhee, S. J., & Rabow, CURRENT Medical Diagnosis & Treatment, 54th ed., vol. 1. McGraw-Hill Education, 2015.
[4] R. Etzioni, N. Urban, S. Ramsey, M. McIntosh, S. Schwartz, B. Reid, J. Radich, G. Anderson, and L. Hartwell, “Early detection: The case for early detection,” Nat. Rev. Cancer, vol. 3, no. 4, pp. 243-252, April 2003.
[5] K. S. H. Yarnall, K. I. Pollak, T. Østbye, K. M. Krause, and J. L. Michener, “Primary Care: Is There Enough Time for Prevention?,” Am. J. Public Health, vol. 93, no. 4, pp. 635-641, April 2003.
[6] D. M. Eddy, “Variations in physician practice: the role of uncertainty,” Health Aff., vol. 3, no. 2, pp. 74-89,1984.
[7] R. Fernandez-Millan, J.-A. Medina-Merodio, R. Plata, J.-J. Martinez-Herraiz, and J.-M. Gutierrez-Martinez, “A Laboratory Test Expert System for Clinical Diagnosis Support in Primary Health Care,” Appl. Sci., vol. 5, no. 3, pp. 222-240, 2015.
[8] A. X. Garg, N. K. J. Adhikari, H. McDonald, M. P. Rosas-Arellano, P. J. Devereaux, J. Beyene, J. Sam,et al, “Effects of Computerized Clinical Decision Support Systems on Practitioner Performance and Patient Outcomes,” JAMA, vol. 293, no. 10, p. 1223, March 2005.
[9] P. Mangiameli, D. West, and R. Rampal, “Model selection for medical diagnosis decision support systems,” Decis. Support Syst., vol. 36, no. 3, pp. 247-259,2004.
[10] G. O. Barnett, J. J. Cimino, J. A. Hupp, E. P. Hoffer, S. E H, G. R. C. P. et al Pryor R A, H. S. S. D. et al, “DXplain,” JAMA, vol. 258, no. 1, p. 67, July 1987.
[11] H. Yan, Y. Jiang, J. Zheng, C. Peng, and Q. Li, “A multilayer perceptron-based medical decision support system for heart disease diagnosis,” Expert Syst. Appl., vol. 30, no. 2, pp. 272-281, 2006.
[12] R. A. Miller, “Medical diagnostic decision support systems—past, present, and future: a threaded bibliography and brief commentary.,” J. Am. Med. Inform. Assoc., vol. 1, no. 1, pp. 8-27, 1994.
[13] J. Basilakis, N. H. Lovell, and B. G. Celler, “A Decision Support Architecture for Telecare Patient Management of Chronic and Complex Disease,” in 2007 29th Annual International Conference of the IEEE Engineering in Medicine and Biology Society, 2007, pp. 4335-4338.
[14] N. H. Lovell, S. J. Redmondl, J. Basilakis2, M. S. Mohktarl, J. A. Sukorl, and B. G. Celler2, “The Application of Decision Support Systems in Home Telecare.”
[15] J. Basilakis, N. H. Lovell, S. J. Redmond, and B. G. Celler, “Design of a Decision-Support Architecture for Management of Remotely Monitored Patients,” IEEE Trans. Inf. Technol. Biomed., vol. 14, no. 5, pp. 1216-1226, September 2010.
[16] H. R. H. Al-Absi, A. Abdullah, and M. I. Hassan, “Soft computing in medical diagnostic applications: A short review,” 2011 Natl. Postgrad. Conf., pp. 1-5, 2011.
[17] K. Kumar, “Type-2 Fuzzy Set Theory in Medical Diagnosis,” vol. 9, no. 1, pp. 35-44, 2015.
[18] Y. Hayashi, “A Neural Expert System with Automated extraction of fuzzy if-then rules and its application in medical diagnosis,” Adv. Neural Inf. Process. Syst. 2, pp. 578-584, 1991
[19] B. G. Celler and R. S. Sparks, “Home telemonitoring of vital signs—technical challenges and future directions.,” IEEE J. Biomed. Heal. informatics, vol. 19, no. 1, pp. 82-91, 2015.
[20] B. Kaplan, “Evaluating informatics applications—clinical decision support systems literature review,” Int. J. Med. Inform., vol. 64, no. 1, pp. 15-37, 2001.
[21] J. G. Chester and J. L. Rudolph, “Vital signs in older patients: age-related changes.,” J. Am. Med. Dir. Assoc., vol. 12, no. 5, pp. 337-43, June 2011.
[22] M. W. Agelink, R. Malessa, B. Baumann, T. Majewski, F. Akila, T. Zeit, and D. Ziegler, “Standardized tests of heart rate variability: normal ranges obtained from 309 healthy humans, and effects of age, gender, and heart rate,” Clin. Auton. Res., vol. 11, no. 2, pp. 99-108, April 2001.
[23] A. Pantelopoulos and N. G. Bourbakis, “A Survey onWearable Sensor-Based Systems for Health Monitoring and Prognosis,” IEEE Trans. Syst. Man, Cybern. Appl. Rev., vol. 40, no. 1, pp. 1-12, 2010.
[24] C. Hacks, “e-Health Sensor Platform V2. 0 for Arduino and Raspberry Pi.” 2015.
[25] C. Novak, “Pediatric Vital Signs Reference Chart PedsCases,” 2016. [Online]. Available: http://www.pedscases.com/pediatric-vital-signs-reference-chart.
[26] Cooking Hacks, “e-Health Sensor Platform V2.0 for Arduino and Raspberry Pi [Biometric Medical Applications],” 2016. [Online]. Available: https://www.cooking-hacks.com/documentation/tutorials/ehealth-biometric-sensor-platform-arduino-raspberry-pi-medical. [Accessed: 10 Aug. 2016].
[27] K. R. Rotheray and G. N. Cattermole, “Rosen's emergency medicine: concepts and clinical practice,” Eur. J. Emerg. Med., vol. 17, no. 2, pp. 101-102, April 2010.
[28] J. S. Hutchison, R. E. Ward, J. Lacroix, P. C. Hébert, M. A. Barnes, D. J. Bohn, P. B. Dirks, S. Doucette, D. Fergusson, R. Gottesman, A. R. Joffe, H. M. Kirpalani, P. G. Meyer, K. P. Morris, D. Moher, R. N. Singh, and P. W. Skippen, “Hypothermia Therapy after Traumatic Brain Injury in Children,” N. Engl. J. Med., vol. 358, no. 23, pp. 2447-2456, June 2008.
[29] Y. K. Axelrod and M. N. Diringer, “Temperature Management in Acute Neurologic Disorders,” Neurol. Clin., vol. 26, no. 2, pp. 585-603, 2008.
[30] K. B. Laupland, “Fever in the critically ill medical patient,” Crit. Care Med., vol. 37, no. Supplement, pp. S273-S278, July 2009.
[31] B. W. Trautner, A. C. Caviness, G. R. Gerlacher, G. Demmler, and C. G. Macias, “Prospective evaluation of the risk of serious bacterial infection in children who present to the emergency department with hyperpyrexia (temperature of 106 degrees F. or higher),” Pediatrics, vol. 118, no. 1, pp. 34-40, July 2006.
[32] Y. Gurevich, “Sequential Abstract State Machines Capture Sequential Algorithms.”, ACM Transactions on Computational Logic, Vol 1, no 1, pp. 77-111, July 2000.
[33] M. Al-Hemairy, M. A. Serhani, S. Amin, M. Alahmad, inventor; A Novel Algorithm for fast disease detection based on vital signs. USA Patent Application no. U.S. 62/377,223, filed on Aug. 19, 2016.

What is claimed is:

1. A system, comprising:
one or more biometric sensors;
one or more processors coupled to the one or more biometric sensors; and
one or more storage devices storing instructions that are operable, when executed by the one or more processors, to cause the one or more processors to perform operations comprising:
obtaining data from the one or more biometric sensors, the data being captured by the one or more biometric sensors during a prior temporal interval;
establishing a weighting factor for each of the one or more biometric sensors;
calculating a control value based on the obtained data;
determining a value of an event indicator based on the obtained data, established weighting factor, and the calculated control value,
accessing a database that correlates corresponding event indicator values with candidate events;
based on the accessed database, establishing a match between the determined event indicator value and a corresponding one of the candidate events; and
performing one or more operations in accordance with the established correspondence.

2. The system of claim 1, wherein the candidate events comprises occurrences of at least one of a medical condition or a disease within a human population.

3. The system of claim 1, wherein the performing comprises:
generating a signal indicative of the established correspondence, the generated signal being indicative of the match between the determined event indicator value and the corresponding candidate event; and
transmitting the generated signal to an additional computing system, the signal instructing the additional computing system to modify a corresponding operational state and present at least one of a graphical, aural, or tactile alert.

4. The system of claim 1, wherein the performing comprises:
generating a graphical representation of the match between the determined event indicator value and the corresponding candidate event; and
presenting the generated graphical representation through a display unit.

5. A computer-implemented method, comprising:
receiving, by at least one processor, data from the one or more biometric sensors, the data being captured by the one or more biometric sensors during a prior temporal interval;
establishing, by the at least one processor, a weighting factor for each of the one or more biometric sensors;
calculating, by the at least one processor, a control value based on the obtained data;
determining, by the at least one processor, a value of an event indicator based on the obtained data, established weighting factor, and the calculated control value,
accessing, by the at least one processor, a database that correlates corresponding event indicator values with candidate events;
based on the accessed database, establishing, by the at least one processor, a match between the determined event indicator value and a corresponding one of the candidate events; and
performing, by the at least one processor, one or more operations in accordance with the established correspondence.

6. A method for detecting a medical condition in a subject, comprising
(a) measuring body conditions using one or more sensors, wherein said sensor(s) is in contact with said subject's body;
(b) defining a weighting factor for each of the sensor;
(c) determining a Control value based on the measured sensor values;
(d) determining an indicator based on the weighting factor, Control value and the measured sensor values; and
(e) searching the value of the indicator in a look-up table to determine a medical condition (disease) corresponding the indicator.
